(19) United States
(12) Patent Application Publication
(10) Pub. No.: US 2007/0191476 A1
(43) Pub. Date: Aug. 16, 2007

(54) Antioxidant Components for Reduction of Nucleic Acid Damage in Companion Animals

(75) Inventors: Paul Richard Heaton, Leicestershire (GB); Brigitte Ester Hope Smith, Leicester (GB); John Merrit Rawlings, Leicester (GB)

Correspondence Address: FULBRIGHT & JAWORSKI, LLP, 1301 MCKINNEY, SUITE 5100, HOUSTON, TX, 77010-3095, US

(73) Assignee: Mars, Incorporated, McClean, VA (US)

(21) Appl. No.: 11/696,865

(22) Filed: Apr. 5, 2007

Related U.S. Application Data
(63) Continuation of application No. 10/722,902, filed on Nov. 26, 2003.
Application No. 10/722,902 is a division of application No. 10/068,697, filed on Feb. 6, 2002, now abandoned.

(30) Foreign Application Priority Data
Aug. 3, 2001 (GB) 0119052.9

(51) Int. Cl. A61K 31/355

(52) U.S. Cl. 514/458; 514/709

(57) ABSTRACT

The present invention is directed to method of using vitamin E, vitamin C and a carotenoid in the manufacture of a foodstuff for reducing nucleic acid damage in a companion animal. The inventions is also directed to a process and a foodstuff for reducing nucleic acid damage in a companion animal that includes the step of feeding the companion animal a foodstuff containing vitamin E, vitamin C and a carotenoid. The process and foodstuff can also include taurine. Preferably the vitamin E is present at a concentration of from 25 IU/400 kcal diet or above, the vitamin C is present at a concentration of from 10 mg/400 kcal or above and the carotenoid is present at a concentration of from 0.01 mg/400 kcal or above. Preferably, the taurine is present at a concentration of from 80 mg/400 kcal or above.

This application is a continuation of U.S. application Ser. No. 10/722,902 filed Nov. 26, 2003, which is a divisional application of U.S. application Ser. No. 10/068,967 filed on Feb. 6, 2002, which claims priority to Great Britain Application No. 0119052.9, which was filed on Aug. 3, 2001.

### TECHNICAL FIELD

The present invention provides nutritional components, for use in reducing nucleic acid damage in a companion animal.

### BACKGROUND OF THE INVENTION

Identifying the mechanisms which are involved in determining species-specific life spans remains one of the outstanding questions of biological aging. Evolution theory proposed that long-lived species are able to provide for their longevity by a more durable soma, including enhanced cellular resistance to stress. Normal cellular processes like respiration and other metabolic activities generate a variety of stresses in the cellular micro environment. These stresses include oxidative stress, heat energy and ionic and pH changes which are produced during normal biochemical reactions, all of which are known to cause damage to cell organelles (e.g., mitochondria, Golgi apparatus, the cytosol, the plasma membrane, the cytoskeleton, lysosomes and the nucleus) and cellular macromolecules (e.g., proteins, polysaccharides, nucleic acids, lipids, phospholipids). Some of the damage caused by these stresses is irreversible.

Accordingly, there is a desire to be able to reduce damage to one or more components of the cellular microenvironment, such as cell organelles or cell macromolecules. The present invention provides nutritional intervention for use in reducing damage to nucleic acid.

### BRIEF SUMMARY OF THE INVENTION

The present invention is directed to method of using vitamin E, vitamin C and a carotenoid in the manufacture of a foodstuff for reducing nucleic acid damage in a companion animal. The inventions is also directed to a process and a foodstuff for reducing nucleic acid damage in a companion animal that includes the step of feeding the companion animal a foodstuff containing vitamin E, vitamin C and a carotenoid. The process and foodstuff can also include taurine.

Preferably the vitamin E is present at a concentration of from 25 IU/400 kcal diet or above, the vitamin C is present at a concentration of from 10 mg/400 kcal or above and the carotenoid is present at a concentration of from 0.01 mg/400 kcal or above. Preferably, the taurine is present at a concentration of from 80 mg/400 kcal or above.

### BRIEF DESCRIPTION OF THE DRAWINGS

For a more complete understanding of the present invention, reference is now made to the following descriptions taken in conjunction with the accompanying drawing, in which:

FIG. 1 shows an effect of varying concentrations of hydrogen peroxide (0-250 μM/ml) on inducing DNA damage. Results are mean±(SEM) of 12 feline subjects. Statistical significance at p<0.001 for means with different letters;

FIG. 2 shows an effect of varying concentrations of hydrogen peroxide (0-250 μM/ml) on inducing DNA damage. Results are mean±SEM of 12 canine subjects. Statistical significance at p<0.001 for means with different letters;

FIG. 3 shows the relationship between visual scoring and computerized image analysis of feline leukocytes for percentage DNA in tail for all classes of DNA damage. Results are mean±SEM (n=100 per class);

FIG. 4 shows the relationship between visual scoring and computerized image analysis of feline leukocytes for tail moment for all classes of DNA damage. Results are mean±SEM (n=100 per class);

FIG. 5 shows the relationship between visual scoring and computerized image analysis of feline leukocytes for tail length for all classes of DNA damage. Results are mean±SEM (n=100 per class);

FIG. 6 shows the relationship between visual scoring and computerized image analysis of canine leukocytes for percentage DNA in tail. Results are mean±SEM (n=100 per class);

FIG. 7 shows the relationship between visual scoring and computerized image analysis of tail moment for all classes of DNA damage for canine leukocytes. Results are mean±SEM (n=100 per class);

FIG. 8 shows the relationship between visual scoring and computerized image analysis of canine leukocytes for tail length for all classes of DNA damage. Results are mean±SEM (n=100 per class);

FIG. 9 shows the endogenous DNA damage in both the control and supplemental groups of cats. Mean values from each group are shown, with standard error mean (SEM) of the means;

FIG. 10 shows the exogenous DNA damage in both the control and supplemented groups of cats. Mean values from each group are shown, with standard error mean (SEM) of the means:

FIG. 11 shows the endogenous DNA damage in both the control and supplemented groups of puppies. Mean values from each group are shown, with standard error mean (SEM) of the means;

FIG. 12 shows the endogenous and exogenous DNA damage in both the control and AOX supplemented groups of dogs taken pre-supplementation. Mean values from each group are shown;

FIG. 13 shows the endogenous and exogenous DNA damage in both the control and AOX-supplemented groups of dogs taken at 2 months post-supplementation. Mean values from each group are shown;

FIG. 14 shows a comparison of the baseline and 2 month post-supplementation endogenous DNA damage results between the no supplement and AOX-supplemented groups of dogs; and

FIG. 15 shows a comparison of the baseline and 2 month post-supplementation exogenous DNA damage results between the no supplement and AOX-supplemented groups of dogs.

### DETAILED DESCRIPTION OF THE INVENTION

Factors which affect cell organelles and cell macromolecules are considered to be wide-ranging. These factors may include environmental influences (temperature pressure), geographical factors, phenotypic factors and nutritional intervention (diet). The present invention has determined, and provides, nutritional intervention for use in reducing damage to the cell macromolecules which are nucleic acid molecules.

Accordingly, the present invention provides the use of vitamin E, vitamin C and a carotenoid in the manufacture of a foodstuff for reducing nucleic acid damage in a companion animal. The nucleic acid may be deoxyribonucleic acid (DNA) or ribonucleic acid (RNA). Yet further, it is contemplated that the term DNA may include nuclear DNA and mitochrondrial DNA.

Vitamin E is a collective term for several biologically similar compounds, including tocopherols and tocotrienols, which share the same biological activity. The most biologically active biological form of vitamin E (also the most active antioxidant) in animal tissue is alpha-tocopherol. Vitamin E cannot be synthesised in vivo. Vitamin E protects against the loss of cell membrane integrity, which adversely alters cellular and organelle function.

Units of vitamin E can be expressed as International Units (IU), where 1 IU of alpha-tocopherol equals 1 mg of alpha-tocopherol. Other vitamin E compounds have their IU determined by their biopotency in comparison to alpha-tocopherol as described in McDowell, L. R (1989) Vitamin E: In vitamins in Animal Nutrition, Chapter 4, page 96, Academic Press, UK.

The vitamin E according to the first aspect of the invention may be in any form. It may be a tocopherol or a tocotrienol. It may be alpha-tocopherol, (d-α or dl-α beta-tocopherol (d-β or dl-β), gamma-tocopherol (d-γ or dl-γ), delta-tocopherol, alpha-tocotrienol, beta-tocotrienol, gamma-tocotrienol or delta-tocotrienol. Preferably it is alpha-tocopherol. The source of the vitamin E is not limiting. Preferred vitamin E sources include vitamin E acetate, (e.g., tocopherol acetate), vitamin E acetate adsorbate or vitamin E acetate spray dried. Preferred sources are synthetic although natural sources may be used. The form of administration of the vitamin E is not limiting. It may be in the form of a diet, foodstuff or a supplement. Hereinafter in this text, the term “foodstuff” covers all of foodstuff, diet and supplement. Any of these forms may be solid, semi-solid or liquid.

The supplement is particularly useful to supplement a diet or foodstuff which does not contain sufficiently high levels of one or more of the components according to the invention. The concentrations of the components in the supplement may be used to “top up” the levels in the animal's diet or foodstuff. This can be done by including a quantity of the supplement with the animal's diet or by additionally feeding the animal a quantity of the supplement. The supplement can be formed as a foodstuff with extremely high levels of one or more components of the invention which requires dilution before feeding to the animal. The supplement may be in any form, including solid (e.g., a powder), semi-solid (e.g., a food-like consistency/gel), a liquid or alternatively, it may be in the form of a tablet or capsule. The liquid can conveniently be mixed in with the food or fed directly to the animal, for example via a spoon, or via a pipette-like device, syringe, etc. The supplement may be high in one or more components of the invention or may be in the form of a combined pack of at least two parts, each part containing the required level of one or more component.

Preferably the vitamin E is incorporated into a commercial petfood product or a commercial dietary supplement. The petfood product may be a dry, semi-dry, a moist or a liquid (drink) product. Moist products include food which is sold in tins or foil containers and has a moisture content of 70 to 90%. Dry products include food which have a similar composition, but with 5 to 15% moisture and presented as biscuit-like kibbles. The diet, foodstuff or supplement is preferably packaged. In this way the consumer is able to identify, from the packaging, the ingredients in the food and identify that it is suitable for the dog or cat in question. The packaging may be metal (usually in the form of a tin or flexifoil), plastic, paper or cardboard. The amount of moisture in any product may influence the type of packaging which can be used or is required.

The foodstuff according to the present invention encompasses any product which a companion animal may consume in its diet. Thus, the invention covers standard food products, as well as pet food snacks (for example snack bars, biscuits and sweet products). The foodstuff is preferably a cooked product. It may incorporate meat or animal derived material (such as beef, chicken, turkey, lamb, blood plasma, marrowbone, etc., or two or more thereof). The foodstuff alternatively may be meat free (preferably including a meat substitute such as soya, maize gluten or a soya product in order to provide a protein source). The product may contain additional protein sources such as soya protein concentrate, milk proteins, gluten etc. The product may also contain a starch source such as one or more grains (e.g., wheat, corn, rice, oats, barely, etc.) or may be starch free. A typical dry commercial dog and cat food contains about 30% crude protein, about 10-20% fat and the remainder being carbohydrate, including dietary fibre and ash. A typical wet, or moist product contains (on a dry matter basis) about 40% fat, 50% protein and the remainder being fibre and ash. The present invention is particularly relevant for a foodstuff as herein described which is sold as a diet, foodstuff or supplement for a cat or dog.

The companion animal of the present invention is not limited. It does not relate to human animals. Companion animals include the domestic cat and the domestic dog, as well as the horse, fish, bird, rabbit and guinea pig. In the present text the terms “domestic” dog and “domestic” cat mean dogs and cats, in particular Felis domesticus and Canis domesticus.

The concentration of vitamin E in a product (solid or liquid or any other form) can easily be determined. For example, it can be determined by HPLC methodology. Preferably, the vitamin E of the foodstuff according to the first aspect of the invention is at a level of 25 IU/400 kcal diet. Throughout this text, references to concentrations per kcal are to kcal total metabolizable energy intake. The determination of calorie density can be identified using Nutritional Requirements of Dogs (1985) National Research Council (U.S.) National Academy Press Washington DC, ISBN: 0-309-03496-5 or Nutritional Requirements of Cats (1986) National Research Council (U.S.) National Academy Press Washington DC, ISBN: 0-309-03682-8. Preferred levels for cats are from 30 IU/400 kcal, from 35 IU/400 kcal, from 40 IU/400 kcal, from 45 IU/400 kcal, from 50 IU/400 kcal, from 55 IU/400 kcal, up to about 100 IU/400 kcal or above. Preferred levels for dogs are from 30 IU/400 kcal, from 40 IU/400 kcal, from 45 IU/400 kcal, from 50 IU/400 kcal, from 55 IU/400 kcal, from 60 IU/400 kcal, from 65 IU/400 kcal, up to about from 100 IU/400 kcal or above.

The first aspect of the invention, also includes vitamin C (ascorbic acid). Vitamin C is a water-soluble substance. It is synthesised de novo in both the domestic cat and the domestic dog. Because it is synthesised in vivo, the effect of vitamin C supplements in dog and cat has not previously been investigated. In particular, the effect of vitamin C supplementation in cat and dog, as a potential antioxidant and in combination with vitamin E supplementation has not been investigated.

The vitamin C according to the first aspect of the invention may be in any form. It may be liquid, semi-solid or solid. Preferably it is a heat stable form such as a form of calcium phosphate. The source of the vitamin C is not limiting. Preferred vitamin C sources include crystalline ascorbic acid (optionally pure), ethylcellulose coated ascorbic acid, calcium phosphate salts of ascorbic acid, ascorbic acid-2-monophosphate salt or ascorbyl-2-monophosphate with small traces of the disphosphate salt and traces of the triphosphate salt, calcium phosphate, or for example, fresh liver. The level of vitamin C in a product (solid, liquid or any other form) can easily be determined. For example, it can be determined by HPLC methodology.

A further useful point in relation to the use of vitamin E in combination with vitamin C is their potential to act synergistically. This may be assisted by the fact that vitamin E is lipid soluble and vitamin C is water-soluble. Alpha-tocopherol is known to sit in the lipid membrane. Ascorbate and alpha-tocopherol, for example, interact at the interface between cell membranes or lipoproteins and water. Ascorbic acid rapidly reduces alpha-tocopherol radicals in membranes to regenerate alpha-tocopherol. The preferred concentration of vitamin C according to the first aspect of the invention is a level which preferably increases the plasma vitamin C level of an animal by up to about 25% (preferably 25% or more) in comparison with when the animal is fed a control diet, such that its total vitamin C consumption is (for both a cat or a dog) 5 mg/400 kcal diet. Levels of vitamin C which do not achieve this increase are still covered by the first aspect of the invention. Levels of vitamin C according to the first aspect of the invention include from 10, 12, 15, 17, 20, 22, 25, 27, 30, 32, 38, 40, 42, 48 up to about 50 mg/400 kcal diet. Preferred levels for the cat are the above options from 10 to 48 mg/400 kcal and for the dog, the above options from 12 to 50 mg/400 kcal. Levels above 55 mg/400 kcal provide no added benefit and are usually best avoided.

The first aspect of the invention also includes a carotenoid. The carotenoids are a group of red, orange and yellow pigments predominantly found in plant foods, particularly fruit and vegetables, and in the tissues of animals which eat the plants. They are lipophilic compounds. Some carotenoids act as a precursors of vitamin A, some cannot. This property is unrelated to their antioxidant activity. Carotenoids can act as powerful antioxidants. Carotenoids are absorbed in varying degrees by different animal species. Carotenoids may be classified into two main groups; those based on carotenes and those based on xanthophylls (which include oxygenated compounds). Common carotenoids include; beta-carotene, alpha-carotene, lycopene, lutein, zeaxanthin and astaxanthin. Carotenoids are not proven to be essential nutrients in the feline or canine diet. Unlike humans and dogs, the cat is unable to convert the precursor beta-carotene into the active vitamin A form since the required enzyme necessary for this conversion is absent from the intestinal mucosa in cats (they do not possess the dioxygenase enzyme which is needed to cleave the carotene molecule).

This invention shows that carotenoids can be absorbed by the domestic cat and dog (to give an increased plasma concentration) and can contribute to a reduction in oxidative stress. Further, the present invention has demonstrated that the carotenoids can be absorbed following their incorporation into a commercial product. As mentioned above, the components of the first aspect of the invention may act synergistically. Vitamin E is able to protect beta-carotene from oxidation and may have a sparing effect on beta-carotene. Vitamin E is thought to protect the chemical bonds of beta-carotene from being oxidized.

The source of the carotenoids is not limiting and can include natural and synthetic sources. In particular, the preferred source is a natural source and includes; marigold meal and lucerne meal (sources of lutein); tomato meal, red palm oil, tomato powder, tomato pomace/pulp (sources of beta-carotene and lycopene). Other sources include, but are not limited to oils high in carotenoid levels and pure manufactured carotenoids such as lutein, violaxanthin, cryptoxanthin, bixin, zeaxanthin, apo-EE (Apo-8-carotenic acid ethylester), canthaxanthin, citranaxanthin, achinenone, lycopene and capsanthin. Preferred levels of total carotenoids are from 0.01 mg/400 kcal, or from 0.2 mg/400 kcal or from 1 mg/400 kcal or from 2 mg/400 kcal.

The concentrations of the following carotenoids are preferably:
Beta-carotene: 0.01 to 1.5 mg/400 kcal, preferably 0.5 to 1 mg/400 kcal
Lycopene: 0.01 to 1.5 mg/400 kcal, preferably 0.5 to 1 mg/400 kcal
Lutein: 0.05 to 1.5 mg/400 kcal, preferably 0.5 to 1 mg/400 kcal.

In particular, the present invention provides for a combination of carotenoids in the first aspect of the invention. Preferred sources of the combined carotenoids include: Red Palm Oil and Marigold Meal; Tomato Powder, Marigold Meal and Lucerne; and Tomato Pomace and Marigold Meal. The level of carotenoid in a product is easily determined. For example, it can be determined by HPLC methodology.

The first aspect of the invention may include taurine. Taurine is an unusual amino acid found in a wide variety of animal species. Taurine is an essential nutrient for the cat which, unlike the dog, is unable to synthesise taurine from precursor amino acids. It is thought that taurine protects cellular membranes from toxic components including oxidants. The increase in vitamin taurine levels in an animal diet can contribute to a reduction in free radicals and therefore a reduction in oxidative stress in the animal, in particular in combination with the other components of the invention. The taurine according to the first aspect of the invention may be in any form, for example, but not limited to powered, crystalline, semi-solid or liquid. The source of the taurine is not limiting. Preferred taurine sources include aminoethylsulfonic acid (C2H7N03S). Sources may be natural or synthetic.

Suitable concentrations of taurine for use according to the first aspect of the invention are usually determined, to some extent as to the processing of the product (for example, whether the product is dry or canned). To maintain plasma taurine levels in the cat at the normal range (>60 μmol/l), a canned (moist) diet must supply at least 39 mg of taurine/kg body weight per day and a dry diet at least 19 mg/kg body weight per day. The first aspect of an invention provides, for a product which is not subjected to a high temperature method (such as canning) a preferred level of from about 80 mg/400 kcal, more preferably from about 100, increasing even more preferably from 120, 150, 180, 200, 220, 250, 280, 300, 320, 350, 400 and above in mg/400 kcal diet. In a product which is processed such as by high temperature, levels according to the invention are preferably from about 380 mg/400 kcal, more preferably from about 400, increasing even more preferably from 420, 450, 480, 500, 520, 550, 580, 600, 620, 650, 700 and above in mg/400 kcal diet. The concentration of taurine in a product (solid liquid or in any other form) can be easily determined. For example, it can be determined by HPLC chromatography.

As described above, the invention includes vitamin E and other components. Useful combinations of the components (preferably in a canned or dry petfood) include;
Vitamin E, vitamin C, taurine, red palm oil and marigold meal
Vitamin E, vitamin C, taurine, tomato powder, marigold meal and lucerne
Vitamin E, vitamin C, taurine, tomato powder and marigold meal
Vitamin E, vitamin C, taurine, tomato powder and lucerne
Vitamin E, taurine, tomato pomace and marigold meal.

A combination of the present invention is;

| Approx. active component | mg/400 kcal after production (Dry Product) | |
|---|---|---|
| Vitamin C | 20 | mg ascorbic acid |
| Vitamin E | 50 | IU |
| Taurine | 200 | mg (500 mg in wet product) |
| Lutein | 0.17 | mg |
| Lycopene | 0.03 | mg |
| Beta-carotene | 0.01 | mg |

A further useful combination of the present invention is:

| | | |
|---|---|---|
| Vitamin E | 50 IU/400 | kcal |
| Vitamin C | 20 mg/400 | kcal |
| Taurine | 500 mg/400 | kcal |
| Beta-carotene | 0.5 to 1 mg/400 | kcal |
| Lycopene | 1 mg/400 | kcal |
| Lutein | 0.5 to 1 mg/400 | kcal |

Other useful components of the foodstuff according to the invention, include; trace minerals (not direct antioxidants, but function as cofactors within antioxidant metalloenzyme systems), selenium (an essential part of the antioxidant selenoenzyme, glutathione peroxidase), copper, zinc and manganese (forming an integral part of the antioxidant metalloenzymes Cu—Zn-superoxide dismutase and Mn-superoxide dismutase.

A second aspect of the invention provides a process for reducing nucleic acid damage in an animal, the process comprising administering a foodstuff comprising vitamin E, vitamin C and a carotenoid to said animal. All preferred features of the first aspect also apply to the second aspect. In accordance with the process of the second aspect, the components may be administered or consumed simultaneously, separately or sequentially.

With increasing evidence suggesting involvement of free radical species in the development of oxidative DNA damage, the consequences of which have been implicated in the etiology of a number of degenerative disorders or diseases the need to accurately assess levels of DNA damage has received renewed attention. Significant levels of DNA damage have been detected in normal human cells, thought to arise from free radical attack (e.g., hydroxyl radicals and other oxidative species) produced as a by-product of normal bodily processes.

A variety of natural defense mechanisms exist to quench or detoxify potentially damaging free radicals. Primary antioxidant defenses include enzymes (e.g., catalase, superoxide dismutase and glutathione peroxidase). Secondary antioxidant defenses may involve excision and repair processes that remove free radical-induced nucleic acid damage. Despite these defense systems damage still occurs within the cell. Thus, it is thought that an accumulation of unrepaired nucleic acids may contribute to a variety of disorders or diseases.

Hydrogen peroxide is believed to be one of the most potent causes of DNA damage, chromosomal alterations and gene mutations by generating highly reactive hydroxyl radicals (OH^{•}) close to the DNA molecule, via the Fenton reaction:
H₂O₂+Fe²⁺→OH^{•}+OH⁻+Fe³⁺

Single-cell electrophoresis, more commonly known as the comet assay, is a simple and very sensitive method for measuring nucleic acid damage (particularly DNA damage) with the added advantage of being able to assess DNA damage at the single-cell level. The basic principle of the assay is that DNA present in all cell types can become damaged, mutated or recombined through the effects of free radical attack. DNA repair enzymes (e.g., DNA endonucleases) remove these damaged sections of DNA. This in effect leaves gaps or “DNA strand breaks” in the DNA. It is these strand breaks that the comet assay is designed to detect and quantify.

To date, the comet assay has been used for a variety of applications, including toxicological studies (Singh et al., 1988), exercise-induced damage (Hartmann et al., 1994), and measuring cell growth and DNA repair mechanisms (Duthie and Collins, 1997). It is important to have the ability to be able to accurately measure levels of free radical damage and how dietary intervention may be able to reduce such damage in cats and dogs. The inventors have developed and validated the comet assay (modified from the original methodology described by Singh et al., (1988)), for measuring levels of oxidative DNA damage (free radical damage) in cat and dog blood samples for inclusion in nutritional studies.

The comet assay works on the principle that free radicals, such as reactive oxygen species, attack and cause DNA strand breaks which leads to unwinding and loss of the DNA supercoil structure. Cells such as leukocytes, are embedded in agarose and layered on a microscope slide, lysed with detergent and electrophoresed under alkaline conditions. Nucleoids are formed, which contain non-nucleosomal but still supercoiled DNA. Any DNA strand breaks present in the DNA cause the supercoiling to relax locally and loops of DNA are then free to extend to form a comet-shaped structure with a distinct “tail” region consisting of stretched and broken DNA loops that have migrated from the nucleoid “head” when subjected to alkaline electrophoresis. The alkaline conditions also allow strands in the broken loops to unwind and convert alkali-labile sites into DNA breaks, to contribute to the formation of the comet “head” and “tail”.

Following fluorescent staining, the intensity of the stain is related to DNA content with DNA damage being quantified by a validated visual grading system and/or computer image analysis package. Two measures of DNA damage are assessed. Firstly, endogenous (background) DNA damage, which gives an indication of naturally occurring DNA strand breaks in the cell. Secondly, artificially induced (cells treated with hydrogen peroxide) DNA damage that reflects antioxidant resistance to exogenous damage. Endogenous and exogenous DNA damage gives an indication that elevated levels of damage (or the elevated stress that causes the damage) contribute to the development of secondary disease.

The comet assay also has proven benefits of:
Requiring only a small blood sample from cats and dogs,
Sensitivity of detecting DNA damage at the single-cell level,
Potentially high-throughput assay,
Ease of application, flexibility and low cost.

The comet assay can be used to discern the different effects of a diet on both endogenous and exogenous DNA damage and consequently can be proposed as a simple bioassay for studying the effects that different nutritional supplements have on modulating levels of DNA damage in cats and dogs.

Although a variety of bodily tissues have been suggested for use in the comet assay, blood leukocytes are considered a good marker of actual bodily state. Leukocytes are more susceptible to the damaging effects of free radicals because of the high percentage of polyunsaturated fatty acids in their plasma membranes and increased production of free radicals as part of their normal function.

The present invention will now be described with reference to the following examples.

### EXAMPLE 1

### Validation of Single-Cell Gel Electrophoresis Assay (Comet Assay) for Assessing Levels of DNA Damage in Canine and Feline Leucocytes

The inventors report herein the development and validation of the comet assay within the canine and feline systems for future use in studying the effects that nutritional supplementation may have on protecting cells from free radical damage.

Materials and Methods

Cell Preparation

All cats and dogs were housed at the Waltham Centre for Pet Nutrition, in conditions resembling those of pet cats and dogs, and were fed commercially available, complete diets throughout the study period. Fasted blood samples (5 ml) were drawn from the jugular vein of 12 healthy adult cats (7.2±4.8 years) and 12 healthy adult dogs (4.5±2.3 years) into lithium herparin vials and diluted 1:1 in PBS. Leukocytes were isolated over Histopaque 1083 gradients (Sigma, UK) by centrifugation at 1000 g for 40 minutes. Leukocytes were washed twice in 10 mls PBS and centrifuged at 700 g for 10 minutes before counting and storing at 1×10⁶ cells/ml in 90% fetal calf serum (Sigma) and 10% dimethyl sulphoxide (Sigma) at −80° C. until required. Viability (assessed by trypan blue exclusion) was typically around 95%.

Hydrogen Peroxide Treatment

DNA damage was induced in vitro by exposing the leukocytes to a range of H₂O₂ concentrations (0-250 μM diluted in PBSa) to determine the optimal level of H₂O₂ required to induce a significant increase in DNA damage above background endogenous DNA damage levels. Leukocytes were thawed rapidly in a 37° C. water bath, washed twice in PBSa, centrifuged at 700 g for 15 minutes and resuspended in PBSa at 2×20⁵/ml. Cells were re-suspended in 0 μM, 10 μM, 50 μM, 100 μM and 250 μM H₂O₂ in PBSa and incubated on ice for 5 minutes. Treated leukocytes were centrifuged at 700 g for 15 minutes at 4° C. ready for slide preparation.

Slide Preparation

Two layers of agarose were prepared. For the first layer, 85 μl 1% (w/v) high-melting point (HMP) agarose (Sigma) prepared at 95° C. in PBSa was pipetted onto fully frosted microscope slides, covered with an 18×18 mm coverslip and allowed to set at 4° C. for 10 minutes. Untreated and hydrogen peroxide-treated leukocytes were washed twice in PBS, centrifuged at 700 g for 15 minutes and resuspended at 2×10⁵ in 85 μl 1% (w/v) low melting point (LMP) agarose (Sigma). The cell suspension was then pipetted over the set HMP agarose layer, covered with an 18×18 mm coverslip and allowed to set at 4° C. for 10 minutes. After the coverslips were removed, the slides were immersed in freshly prepared cold lysis solution.

Cell Lysis

Slides were immersed in pre-chilled lysis solution (2.5 M NaCl, 100 mM sodium EDTA, 10 mM Tris, pH adjusted to 10 using NaOH pellets, 1% Triton X-100 (v/v), (added immediately before use)) for 60 minutes at 4° C. in order to remove cellular proteins.

Alkaline Treatment and Electrophoresis

Following lysis, the slides were placed in a gel electrophoresis unit and incubated in fresh alkaline electrophoresis buffer (300 mM NaOH, 1 mM EDTA, pH 13) for 40 minutes at room temperature in the dark, before being electrophoresed at 25V (300 mA) for 30 minutes at 4° C. in the dark.

Neutralization and Staining

Following electrophoresis, the slides were immersed in neutralization buffer (0.4M Tris-HCl, pH 7.5) and gently washed three times for 5 minutes at 4° C. to remove alkalis and detergents. Fifty μl of SYBR Green (Trevigen, Gathersberg, Md.) were added to each slide to stain the DNA, then covered with a coverslip and kept in the dark in an air-tight moist container before viewing. SYBR Green was chosen for staining damaged DNA following studies by Ward & Marples (2000), demonstrating improved detection sensitivity and assay resolution of SYBR Green over alternative DNA stains.

Scoring for DNA Damage

Visual and computerized image analysis of DNA damage was carried out in accordance with the protocols of Collins et al., (1996, 1997). Slides were examined at 250× magnification on a Zeiss inverted fluorescence microscope at 460 nm. Randomly selected non-overlapping cells were visually assigned a score on an arbitrary scale of 0-4 (i.e. ranging from 0=no DNA damage, to 4=extensive DNA damage) based on perceived comet tail length migration and relative proportion of DNA in the comet tail. A total damage score for each slide was derived by multiplying the number of cells assigned to each grade of damage by the numeric value of the grade and summing over all grades (giving a maximum possible score of 400, corresponding to 100 cells at grade 4). To determine whether visual scoring correlated with computerized image analysis the same cells were also scored for DNA damage using the KOMET 4.0 analysis package (Kinetic Imaging, Liverpool, UK). A variety of objective measurements including, percentage DNA in tail, tail length (measured from the leading edge of the comet head), and tail moment were made. Tail moment was calculated as follows:
Tail moment=Tail length×% Tail DNA/100
Statistical Analysis

Linear regression analysis was used to correlate visual comet scores with computerized image analysis derived scores. A two-factor ANOVA as well as the Student-Newman-Keuls test were used in order to determine statistically significant differences between the different concentrations of H₂O₂ used to induce in vitro DNA damage.

Results

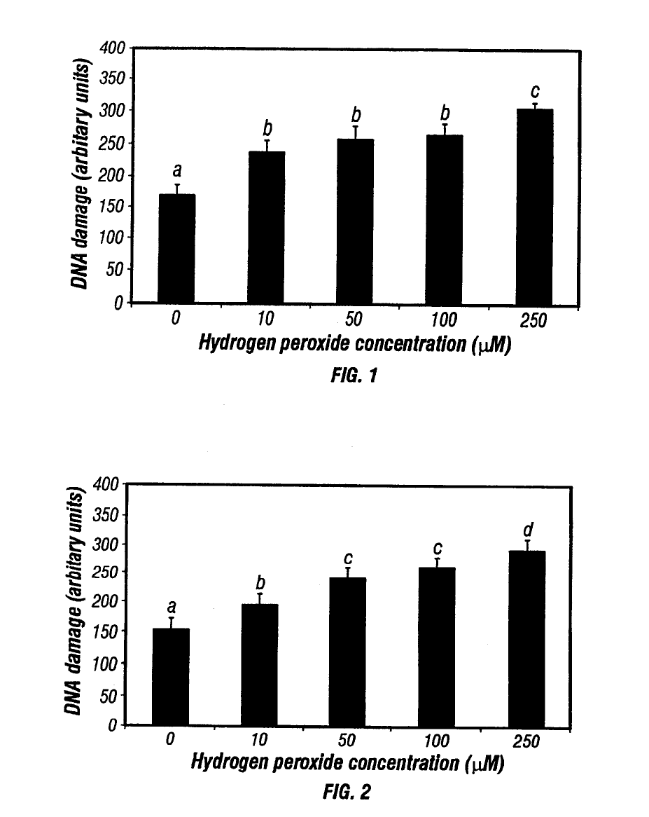

The objective of the present study was to develop and validate the use of the comet assay for assessing levels of DNA damage in feline and canine leukocytes. DNA damage is scored visually from class 0 (no DNA damage) to class 4 (extensive DNA damage) using perceived comet tail length and level of DNA in the tail as the scoring criteria. To demonstrate the susceptibility of feline and canine leukocytes to DNA damage, suspensions of cells were treated for 5 minutes with 0-250 μM H₂O₂. SYBR green-stained comets were then assessed for DNA damage using the visual scoring system. Statistically significant increases in DNA damage (p<0.001) were observed over the range of 10-250 μM H₂O₂ in both feline and canine samples when compared to untreated samples using the visual scoring system. While use of 250 μM H₂O₂ induced significant increases in DNA damage in relation to all other concentrations of H₂O₂ used in both canine and feline samples (FIGS. 1 and 2), no significant differences were observed between the levels of DNA damage when comparing use of 10-100 μM H₂O₂ with the feline samples (FIG. 1) and 50-100 μM H₂O₂ with the canine samples (FIG. 2).

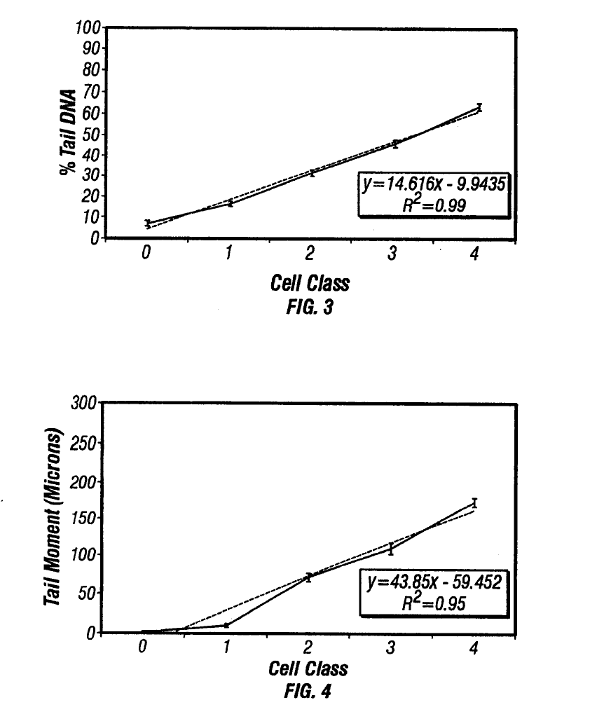

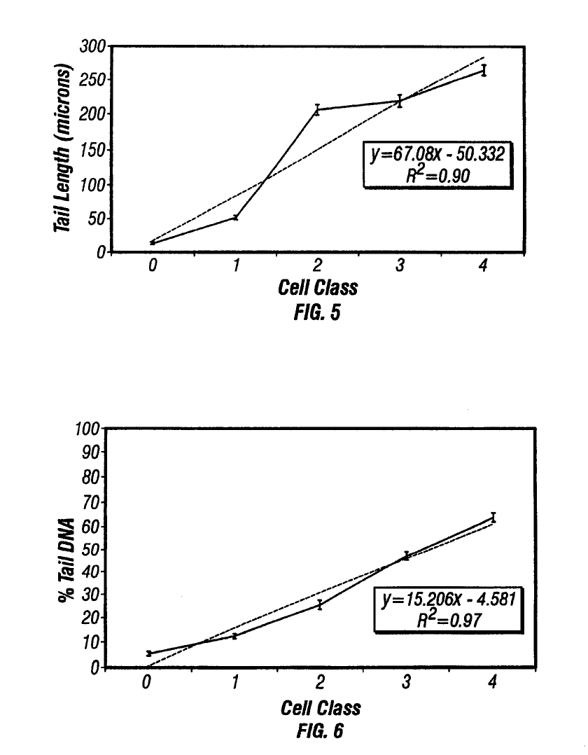

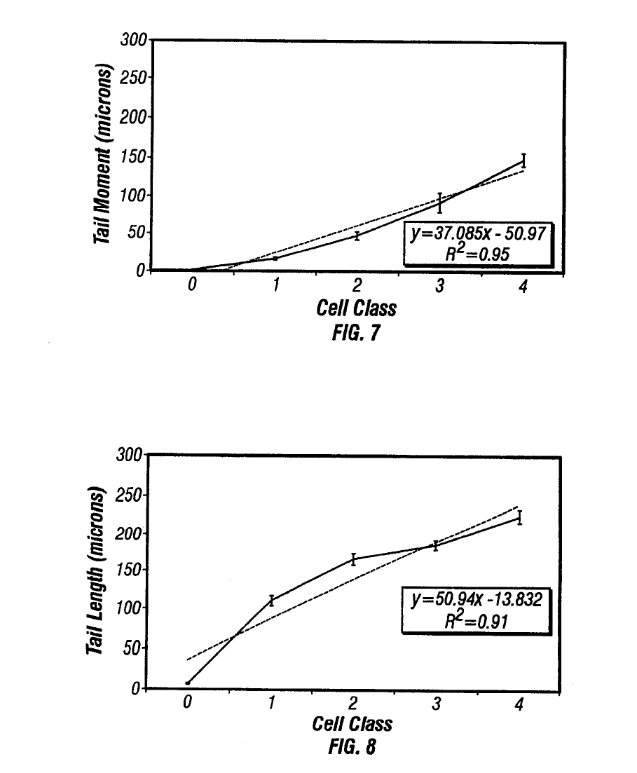

The second objective of this study was to compare visual scoring of comets (on a scale of 0-4) with computerized image analysis parameters of percentage DNA in tail, tail moment and tail length. FIGS. 3, 4 and 5 show that visual scoring of feline leukocyte comets were highly correlated with computer image analysis, as determined by linear regression, for percentage DNA in tail (R²>0.99), tail moment (R²>0.95) and tail length (R²>0.90), respectively A similar trend was also observed when correlating the visual and computer image analysis of canine leukocyte comets, percentage DNA in tail (R²>0.97), tail moment (R²>0.95) and tail length (R²>0.91), FIGS. 6, 7 and 8, respectively.

### EXAMPLE 2

### Assessing Levels of DNA Damage in Antioxidant Supplemented Versus Control Cats Using the Comet Assay

Animals

All cats were housed at the Waltham Centre for Pet Nutrition, in conditions resembling those of pet cats. The test control group consisted of 14 adult domestic shorthaired cats (9.2±2.1 years) and were maintained on a commercially available complete diet. The antioxidant supplemented group of 14 adult domestic shorthaired cats (8.7±1.9 years) were maintained on the same commercial canned diet which additionally contained the following antioxidant supplements (Table 1). All cats had been on their respective diets for over 2 years.

TABLE 1
levels of the Components of the antioxidant
cocktail present in wet diet.

| Ingredient | mg/400 kcal |
|---|---|
| α-tocopherol | 50 |
| Ascorbate | 40 |
| β-carotene | 0.5 |
| Lutein | 0.5 |
| Taurine | 500 |
| Lycopene | 0.7 |

Sample Collection

Whole blood specimens were collected into a 5 ml lithium heparin tube. The leukocyte cell fraction was then purified and separated from the whole blood for comet analysis.

Comet Assay

The comet assay was performed as discussed in Example 1.

Results

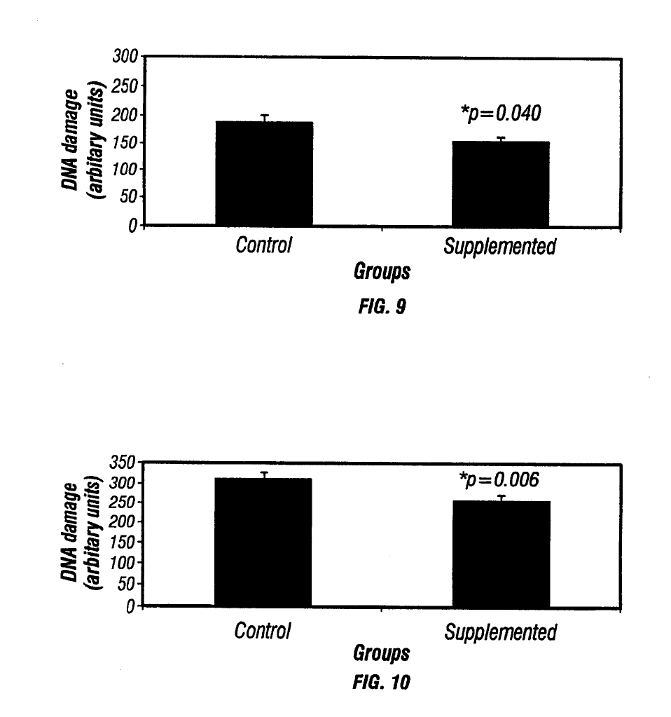

These results shown in FIGS. 9 and 10 demonstrate a significant reduction in levels of endogenous and exogenous DNA damage in the supplemented group of cats compared to the non-supplemented group of control cats. This demonstrates significantly higher antioxidant resistance in the supplemented cats, leading to reduced susceptibility and exposure of DNA to endogenous and exogenous free radical attack, reducing the damage that potentiates DNA instability, mutation and dysfunction.

Endogenous DNA damage gives an indication that elevated levels of damage (or the elevated oxidative stress that causes the damage) contributes to the development of secondary diseases. This approach can be applied to the progression of degenerative disorders. In addition, DNA damage and mutation may result in:
(a) Failure of immunological cells to proliferate because of DNA-damage mediated cell-cycle arrest,
(b) Decreased rates of proliferation, as a consequence of selection in vivo against cells carrying certain mutations will lead to sub-optimal immune responses to infection,
(c) Increased levels of apoptosis, triggered by critical levels of DNA damage will lead to reduced numbers of immunological cell-types.

Thus, reduction of endogenous and exogenous DNA damage levels through antioxidant supplementation in cats, may indicate reduced susceptibility to degenerative disorders, through reducing the susceptibility of DNA to free radical damage as well as possibly increasing the levels of DNA repair.

### EXAMPLE 3

### Assessing Levels of DNA Damage in Antioxidant Supplemented Versus Control Puppies Using the Comet Assay

Two groups of four, age and sex matched, Labrador retriever littermates were maintained to body weight on a complete balanced diet with supplements adjusted accordingly from 6 weeks of age until sampling for the comet assay at 15 months of age. One group was supplemented with an antioxidant cocktail, the ingredients of which are given in Table 2.

TABLE 2
Levels of the components of the cocktail.

| Ingredient | mg/400 kcal |
|---|---|
| α-tocopherol | 50 |
| Ascorbate | 40 |
| β-carotene | 0.5 |
| Lutein | 0.5 |
| Taurine | 500 |

Sample Collection

Whole blood specimens were collected into a 5 ml lithium heparin tube. The leukocyte cell fraction was then purified and separated from the whole blood for comet analysis.

Comet Assay

The comet assay was performed as discussed above in Example 1.

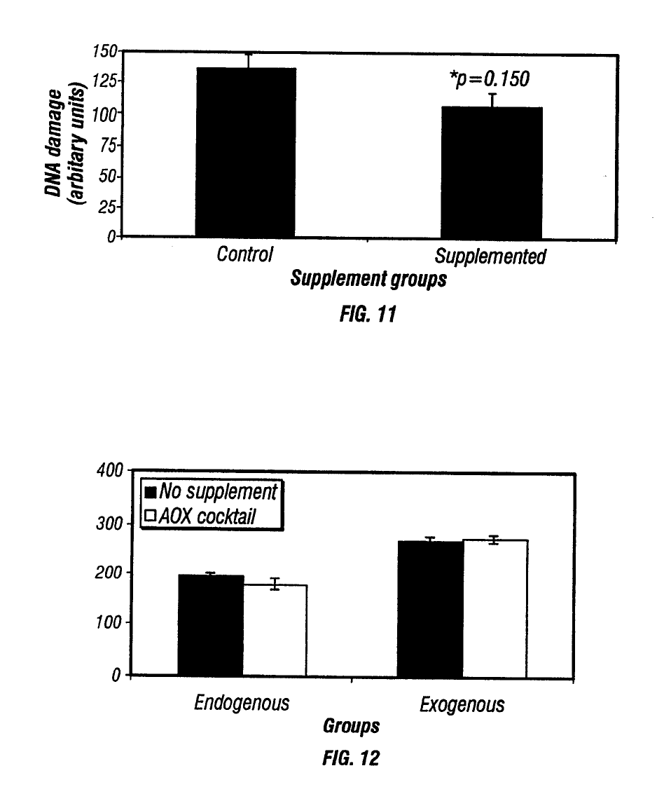

The results shown in FIG. 11 demonstrate a reduction in levels of endogenous DNA damage in the supplemented group of puppies (p=0.150) compared to non-supplemented group of control puppies.

Thus, reduction of endogenous DNA damage levels through supplementation in puppies, indicate reduced susceptibility to infection and degenerative disorders, including the ageing process in general, through reducing the susceptibility of DNA to free radical damage as well as possibly increasing the levels of DNA repair.

### EXAMPLE 4

### Assessing Levels of DNA Damage in Supplemented Versus Control Adult Dogs Using the Comet Assay

Two groups of 20, age and sex matched adult dogs of mixed breed were maintained to body weight on a complete balanced diet with supplements adjusted accordingly for a 6 month period. Sampling for the comet assay was carried out on a monthly basis. One group of dogs was supplemented with an antioxidant cocktail, the ingredients of which are given in Table 3.

TABLE 3
Levels of the components of the cocktail.

| Ingredient | mg/400 kcal |
|---|---|
| α-tocopherol | 50 |
| Ascorbate | (20) 40 |
| β-carotene | 0.5 |
| Lutein | 0.5 |
| Taurine | (200) 500 |
| Lycopene | 0.7 |

(The bracketed figures refer to concentration in dry diet format.)

Sample Collection

Whole blood specimens were collected into a 5 ml lithium heparin tube. The leukocyte cell fraction was then purified and separated from the whole blood for comet analysis.

Comet Assay

The comet assay was performed as discussed in Example 1.

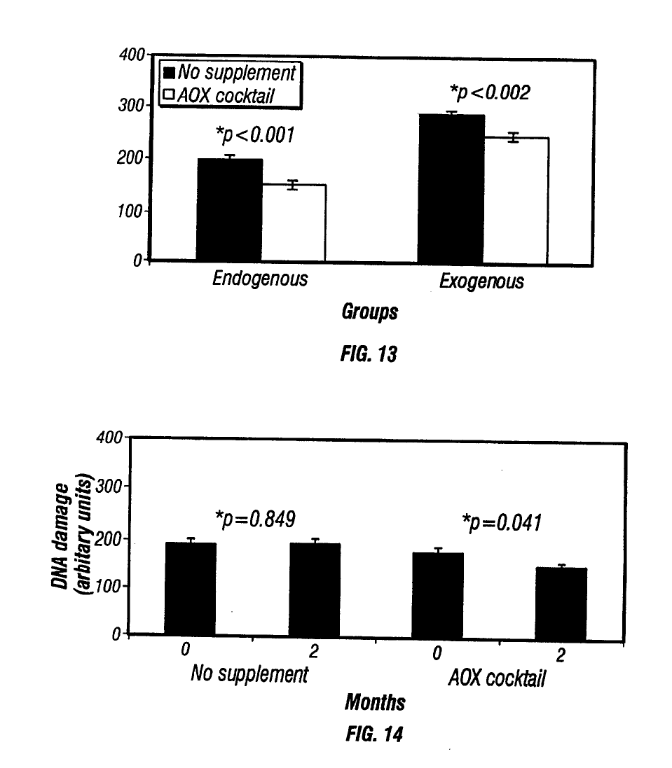

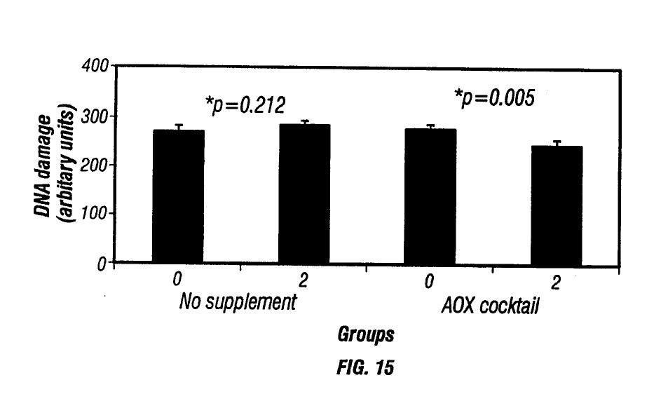

The results shown in FIGS. 12 to 15 demonstrate a significant reduction in levels of both endogenous (p=0.001) and exogenous (p=0.003) DNA damage in the AOX-supplemented group of dogs at 2 months post-supplementation, compared to the non-supplemented group of control dogs (FIG. 13). No significant differences were noted in endogenous or exogenous DNA damage levels between the two groups at baseline (FIG. 12). Also the control group showed no significant change in either endogenous or exogenous levels of DNA damage when comparing samples taken at 2 months post-supplementation to baseline levels (FIGS. 14 and 15). However, when the 2 month supplementation levels of exogenous and endogenous DNA damage from the AOX-supplemented group of dogs were compared to their baseline values there were significant reductions in endogenous DNA damage (p=0.041; FIG. 14) and exogenous DNA damage (p=0.005; FIG. 15).

What is claimed is:

1. A method of producing a foodstuff for reducing nucleic acid damage in a companion animal comprising the step of adding vitamin E, vitamin C and a carotenoid to the foodstuff.

2. The method of claim 1, further including taurine.

3. The method of claim 1, wherein the carotenoid is one or more of beta-carotene, lutein or lycopene.

4. The method of claim 1, wherein the vitamin E is present at a concentration of from 25 IU/400 kcal diet or above.

5. The method of claim 1, wherein the vitamin C is present at a concentration of from 10 mg/400 kcal or above.

6. The method of claim 1, wherein the carotenoid is present at a concentration of from 0.01 mg/400 kcal or above.

7. The method of claim 2, wherein the taurine is present at a concentration of from 80 mg/400 kcal or above.

8. The method of claim 1, wherein the foodstuff is selected from a group consisting of dry, wet, and semi-dry foodstuff and a supplement.

9. The method of claim 1, wherein the companion animal is selected from a group consisting of a cat, dog, horse, fish, bird, rabbit and guinea pig.

10. A process for reducing nucleic acid damage in a companion animal comprising the step of feeding the companion animal a foodstuff containing vitamin E, vitamin C and a carotenoid.

11. The process of claim 10, wherein the nucleic acid damage is DNA damage.

12. The process of claim 10, wherein the foodstuff further contains taurine.

13. The process of claim 10, wherein the carotenoid is one or more of beta-carotene, lutein or lycopene.

14. The process of claim 10, wherein vitamin E is present at a concentration of from 25 IU/400 kcal diet or above.

15. The process of claim 10, wherein vitamin C is present at a concentration of from 10 mg/400 kcal or above.

16. The process of claim 10, wherein carotenoid is present at a concentration of from 0.01 mg/400 kcal or above.

17. The process of claim 12, wherein taurine is present at a concentration of from 80 mg/400 kcal or above.

18. The process of claim 10, wherein the components are administered simultaneously, separately or sequentially.

19. The process of claim 10, wherein the companion animal is selected from a group consisting of a cat, dog, horse, fish, bird, rabbit and guinea pig.
